(19) United States
(12) Patent Application Publication
(10) Pub. No.: US 2004/0199179 A1
(43) Pub. Date: Oct. 7, 2004

(54) Steerable ablation probe

(76) Inventor: Christopher J. Elliott, Hopkinton, MA (US)

Correspondence Address: BINGHAM, MCCUTCHEN LLP, THREE EMBARCADERO, SUITE 1800, SAN FRANCISCO, CA, 94111-4067, US

(21) Appl. No.: 10/406,068

(22) Filed: Apr. 2, 2003

(51) Int. Cl. A61B 17/22

(52) U.S. Cl. 606/128; 606/127; 604/19

(57) ABSTRACT

A probe assembly and method for ablating tissue is provided. The probe assembly comprises an elongated shaft having a rigid section and a flexible section distally extending from the rigid section. The probe assembly further comprises a tissue penetrating element associated with the flexible section. For example, it can be mounted to the flexible section or can be located on a separate element, such as a trocar that can be disposed within the elongated shaft. The probe assembly further comprises one or more ablative elements, such as electrodes, that is associated with the flexible section. Like the tissue penetrating element, the ablative element(s) can be mounted to the flexible section or can be located on a separate element. Thus, the flexible section of the shaft can be flexed to deflect the tissue penetrating element, so that it can be steered through tissue. In this manner, sensitive anatomical structures can be avoided, e.g., if the sensitive structure lies between the tissue region to be treated and the entry point of the probe assembly. An optional steering assembly can be used to provide active steering to the probe assembly.

### FIELD OF THE INVENTION

The field of the present invention relates generally to the structure and use of radio frequency (RF), electro-surgical probes for the treatment of tissue disorders, such as tumors and lesions.

### BACKGROUND OF THE INVENTION

Radio frequency (RF) energy can be used to ablate solid tissue, thus inducing localized tissue necrosis. RF energy is particularly useful in this capacity, for inducing necrosis in sub-dermal lesions and tumors, such as those found in cancers of the liver, stomach, kidney, lung, bowel, and pancreas. The conventional delivery system for this sort of treatment is an electro-surgical probe that can be percutaneously or laparoscopically introduced into the patient's body and advanced through tissue to reach the pathology.

A typical electro-surgical ablation probe includes one or more tissue penetrating needle electrodes, which when coupled to an RF generator, emit RF energy from the exposed, uninsulated portion of the electrode(s). This energy translates into ion agitation, which is converted into heat and induces cellular death via coagulation necrosis. These types of ablation probes typically have a rigid construction, so that they can be advanced through tissue without axially collapsing. While this aids in tissue penetration, rigid ablation probes are better suited to reaching anatomical locations that are directly accessible via a straight-line approach from outside the body. If the anatomical location resides adjacent a sensitive structure, such as an organ or blood vessel, a straight-line approach may risk damage to this structure during the probe insertion process, especially if the sensitive structure lies between the tissue to be treated and the entry point of the ablation probe.

Consequently, there is a significant need for an ablation probe that can be used to ablate a pathology without significant risk of damaging sensitive tissue that resides between the pathology and the entry point of the ablation probe.

### SUMMARY OF THE INVENTION

In accordance with a first aspect of the present inventions, a probe assembly for ablating tissue is provided. The probe assembly comprises an elongated shaft having a rigid section and a flexible section distal to the rigid section. The rigid section can be composed of any material that provides it with columnar strength, e.g., a suitable metal or plastic. The flexible section can have any arrangement that allows it to laterally flex. For examples, the flexible section may comprise a relatively short polymeric tubular structure, a plurality of segmented rigid elements, or a polymeric tubular structure that is axially reinforced with one or more stiffening members, such as leaf springs.

The probe assembly further comprises a rigid tissue penetrating element associated with the flexible section of the shaft. In one embodiment, the tissue penetrating element can be distally mounted to the flexible section. In another embodiment, the tissue penetrating element can be separate from the elongated shaft. For example, the probe assembly can further comprise a trocar reciprocatably disposed within the cannula, in which case, the tissue penetrating element can be disposed on the distal end of the trocar. By way of non-limiting example, the tissue penetrating tip allows the probe assembly to be introduced through tissue, such as skin, underlying fascia, and tough tissue where tumors may be located. The tissue penetrating tip also allows penetration of unstable tumors that require a quick stabbing motion. Although the present inventions should not be necessarily limited thereby in their broadest aspects, the flexible section allows the tissue penetrating element to deflect relative to the rigid section, e.g., so that the tissue penetrating tip can be steered through tissue. The tissue penetrating tip can optionally be faceted for ultrasound visualization.

The probe assembly further comprises one or more ablative elements associated with the flexible section of the shaft. For example, the ablative element(s) can be disposed on the tissue penetrating element or can be distally deployable from the shaft. The ablative element(s) can be any element that ablates tissue, e.g., laser or chemical releasing element, but in the preferred embodiment, the ablative element(s) takes the form of one or more electrodes, and specifically, needle electrode(s).

The probe optionally comprises a steering mechanism for actively flexing the flexible section of the shaft. The steering mechanism may be configured to flex the flexible section of the shaft in a single direction or multiple directions. In one embodiment, the steering mechanism comprises one or more wires mounted to the flexible section of the shaft or the tissue penetrating element. In another embodiment, the steering mechanism comprises shape-memory linkages mounted to the flexible section of the shaft or the tissue penetrating element.

In accordance with another aspect of the present invention, a method of treating a tissue region within a patient is provided. In many cases, the tissue region may be a tumor, but the method can have any application where tissue can be treated via ablation, e.g., soft-tissue ablation in orthopedics, pain-management (e.g., spinal disk shrinkage), trans-vaginal ablation of uterine fibroids, fallopian tube closure for sterilization, etc. The method comprises inserting a medical probe into the body of the patient via an entry point. The medical probe can be, e.g., inserted percutaneously, laparoscopically, or even through a surgical opening. The method further comprises identifying a sensitive anatomical structure between the entry point and the tissue region. For example, the sensitive structure can be an organ or a blood vessel.

The method further comprises advancing the medical probe, such that the distal end of the medical probe bypasses the sensitive anatomical structure, and bending the distal end of the medical probe while the medical probe is within the body of the patient. In the preferred method, the distal end of the medical probe is bent after bypassing the sensitive anatomical structure, but can be bent prior to bypassing the sensitive anatomical structure depending upon the particular situation. In a non-limiting method, the medical probe is initially advanced, such that the distal end of the medical probe is adjacent an initial target site, and then, after the distal end of the medical probe has been bent, advanced again, such that the distal end is adjacent a final target site.

The method further comprises placing one or more ablative elements associated with the distal end of the medical probe adjacent the tissue region (e.g., by embedding the ablative element(s) within the treatment region), and ablating the tissue region with the ablative element(s). For example, if the ablative element(s) are affixed to the distal end of the medical probe, they are simply placed adjacent the treatment region. If the ablative element(s) are initially housed with the medical probe, they can be deployed therefrom. In the preferred methods, the tissue region is ablated using RF energy. Other types of ablative techniques can be used, including laser energy, microwave energy, and chemical solutions.

### BRIEF DESCRIPTION OF THE DRAWINGS

The drawings illustrate the design and utility of a preferred embodiment of the present invention, in which similar elements are referred to by common reference numerals. In order to better appreciate the advantages and objects of the present invention, reference should be made to the accompanying drawings that illustrate this preferred embodiment. However, the drawings depict only one embodiment of the invention, and should not be taken as limiting its scope. With this caveat, the invention will be described and explained with additional specificity and detail through the use of the accompanying drawings in which:

FIG. 1 is a plan view of a tissue ablation system constructed in accordance with one preferred embodiment of the present invention;

FIG. 2 is a partial cutaway, cross-sectional view of a probe assembly that can be used in the tissue ablation system of FIG. 1, wherein the needle electrode array is particularly shown retracted within the probe assembly;

FIG. 2A is a cross-sectional view of the probe assembly of FIG. 2, taken along the line 2A-2A;

FIG. 3 is a partial cutaway, cross-sectional view of the probe assembly of FIG. 2, wherein the needle electrode array is particularly shown deployed from the probe assembly;

FIG. 4 is partially cutaway side view of the probe assembly of FIG. 2, particularly showing the tissue penetrating tip deflected in one direction;

FIG. 5 is partially cutaway side view of the probe assembly of FIG. 2, particularly showing the tissue penetrating tip deflected in another direction;

FIG. 6 is a partial cutaway, cross-sectional view of another probe assembly that can be used in the tissue ablation system of FIG. 1;

FIG. 6A is a cross-sectional view of the probe assembly of FIG. 6, taken along the line 6A-6A;

FIG. 7 is partially cutaway side view of the probe assembly of FIG. 6, particularly showing the tissue penetrating tip deflected in one direction;

FIG. 8 is partially cutaway side view of the probe assembly of FIG. 6, particularly showing the tissue penetrating tip deflected in another direction;

FIG. 9 is a partial cutaway, cross-sectional view of still another probe assembly that can be used in the tissue ablation system of FIG. 1;

FIG. 9A is a cross-sectional view of the probe assembly of FIG. 9, taken along the line 9A-9A;

FIG. 10 is partially cutaway side view of the probe assembly of FIG. 9, particularly showing the tissue penetrating tip deflected in one direction;

FIG. 11 is partially cutaway side view of the probe assembly of FIG. 9, particularly showing the tissue penetrating tip deflected in another direction;

FIG. 12 is a partial cutaway, cross-sectional view of yet another probe assembly that can be used in the tissue ablation system of FIG. 1;

FIG. 13 is a partial cutaway, cross-sectional view of still yet another probe assembly that can be used in the tissue ablation system of FIG. 1;

FIG. 13A is a cross-sectional view of the probe assembly of FIG. 13, taken along the line 13A-13A; and

FIGS. 14A-14E are cross-sectional views of one preferred method of using the tissue ablation system of FIG. 1 to treat tissue.

### DETAILED DESCRIPTION OF THE PREFERRED EMBODIMENTS

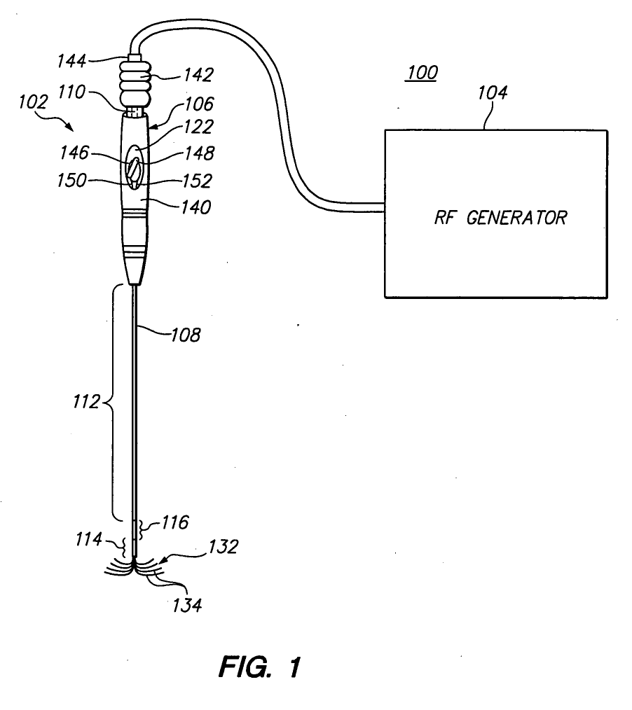

FIG. 1 illustrates a tissue ablation system 100 constructed in accordance with a preferred embodiment of the present invention. The tissue ablation system 100 generally comprises an ablation probe assembly 102, which is configured for introduction into the body of a patient to ablate target tissue, such as a tumor, and a radio frequency (RF) generator 104 configured for supplying RF energy to the probe assembly 102 in a controlled manner.

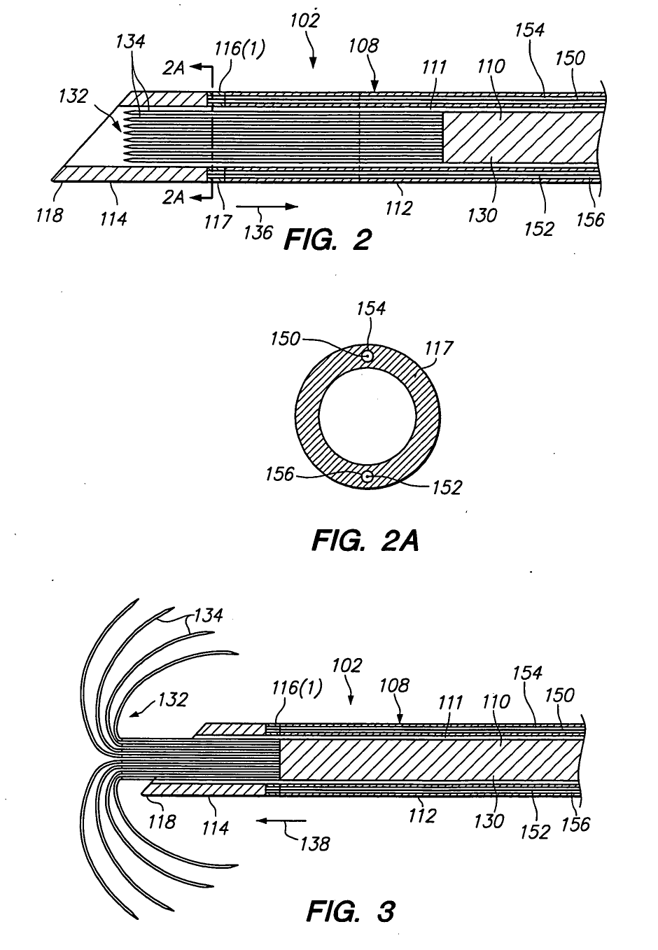

Referring further to FIGS. 2 and 3, the probe assembly 102 generally comprises a steerable handle assembly 106, an elongated cannula 108, and an inner probe 110 slideably disposed within the cannula 108. As will be described in further detail below, the cannula 108 serves to deliver the active portion of the inner probe 110 to the target tissue. The cannula 108 has a suitable length, typically in the range of 5 cm to 30 cm, preferably from 10 cm to 20 cm. The cannula 108 has an outside diameter consistent with its intended use, typically being from 1 mm to 5 mm, usually from 1.3 mm to 4 mm. The cannula 108 has an inner diameter in the range of 0.7 mm to 4 mm, preferably from 1 mm to 3.5 mm.

The cannula 108 comprises a central lumen 111 through which the inner probe 110 is slidably disposed. The cannula 108 has sufficient columnar strength, such that it can penetrate and be advanced through tissue, yet provides steerability to the probe assembly 102. To this end, the cannula 108 comprises a rigid section 112, a rigid tissue penetrating tip 114, and an intermediate flexible section 116 mounted between the rigid section 112 and a penetrating tip 114. The rigid section 112 and tissue penetrating tip 114 are composed of a suitable material, such as plastic or metal. The tissue penetrating tip 114 has a sharp point 118 that is created by beveling the distal end of the tissue penetrating tip 114.

It is noted that the use of a sharp tissue penetrating tip 114 allows the cannula 108 to penetrate through skin and underlying fascia, thereby facilitating a percutaneous introduction procedure. The use of a sharp tissue penetrating tip 114 has other advantages as well. The sharp tissue penetrating tip 114 allows the cannula 108 to be introduced into tough tissue where tumors may be found. For example, in the case of hepatocellular carcinoma (HCC), the liver is very cirrhotic, with a tough, fibrous nature that requires a sharp tip for continued penetration to the tumor. The sharp tissue penetrating tip 114 also allows penetration of tumors that are unstable within the surround tissue. For example, breast tumors, in which there is an increasing interest for ablation, have been likened to a “golf ball in a gelatin.” In this case, accurate targeting of a breast tumor by an ablation probe requires a quick, accurate stab with a sharp tip. Lung tumors have been described with similar properties.

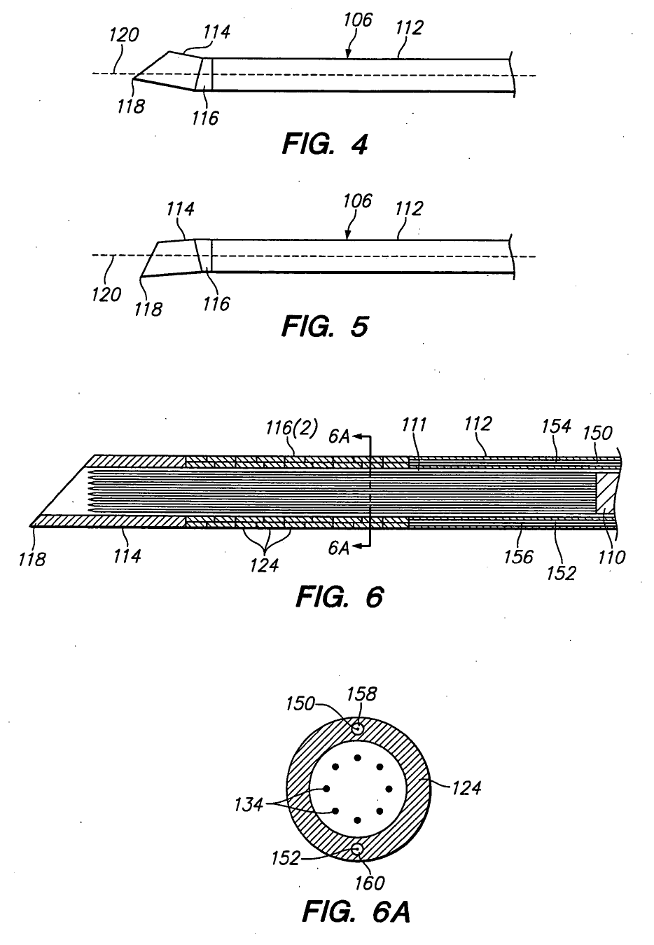

In the embodiment illustrated in FIGS. 2 and 3, a specific embodiment of a flexible section 116(1) comprises a tubular structure 117 composed of a suitable polymeric material that can tolerate both tensile and compressive forces, such as, e.g., polyurethane or silicone. The tubular structure 117 is bonded between the rigid section 112 and tissue penetrating tip 114 using a suitable biocompatible adhesive material, such as, e.g., cyanoacrylate, uv-curable adhesives, or RTV silicone. The tubular structure 117 could also be incorporated via insert molding or potting. Thus, it can be appreciated that the flexible section 116(1) allows for angulation between the tissue penetrating tip 114 and the rigid section 112, as illustrated in FIGS. 4 and 5. In the preferred embodiment, the angle between the maximum deflection of the tissue penetrating tip 114 and the longitudinal axis 120 of the rigid section 112 is within the range of 5-10°. It should be noted that lesser or greater deflection angles can be achieved, depending upon the desired steering capability. In any event, the length of the flexible section 116(1) is relatively short to prevent it from axially collapsing upon itself, thereby allowing better control over the tissue penetrating tip 114 as the cannula 108 is advanced through tissue. As will be described in further detail below, the handle assembly 106 comprises a steering mechanism 122 that facilitates control over the angle and the direction of the tissue penetrating tip 114.

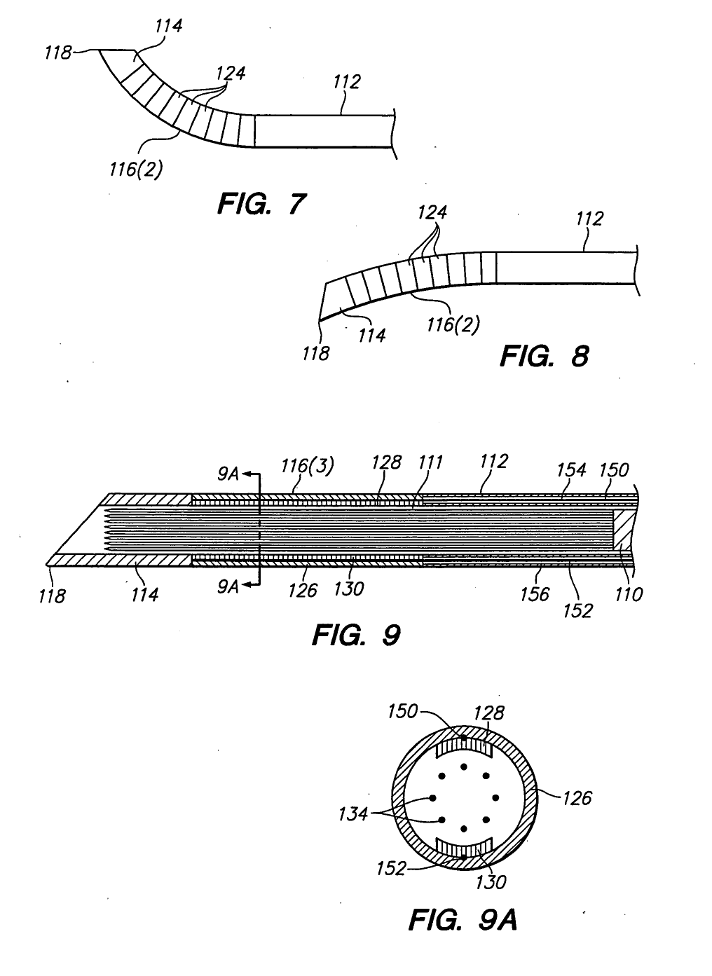

Referring to FIGS. 6-8, an alternative intermediate flexible section 116(2) can be used to facilitate deflection of the tissue penetrating tip 114. In this embodiment, the flexible section 116(2) comprises a plurality of rigid ring shaped segments 124 that can angularly move relative to each other (as illustrated in FIGS. 7 and 8), yet provide no or very little axial movement relative to each other. As can be appreciated, the length of the flexible section 116(2) can be relatively long, since the rigid segments 124 provide the necessary columnar strength.

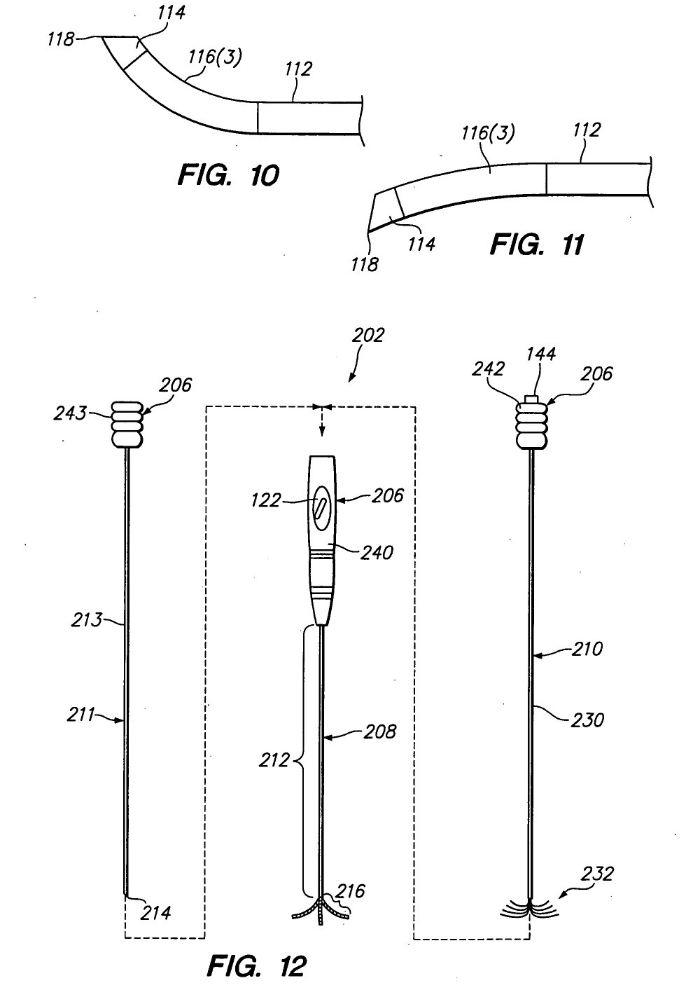

Referring to FIGS. 9-11, another alternative intermediate flexible section 116(3) can be used to facilitate deflection of the tissue penetrating tip 114. In this embodiment, the flexible section 116(3) comprises a flexible polymeric tube 126. In order to provide columnar strength, the flexible section 116(3) is reinforced with a pair of resilient flat stiffening members 128 and 130, and specifically leaf springs, that extend along opposite sides of the flexible tube 126. Thus, the leaf springs 128 and 130 allow the flexible section 116(3) to laterally flex, while preventing it from collapsing in the presence of an axial force.

Even more alternatively, the intermediate flexible section can be composed of a polymeric extruded tube with variable stiffness properties along the length of the flexible section. This can be accomplished by, e.g., using an interrupted-layer extrusion that effects this variation within one extrusion session; i.e., there is no need to bond a less-stiff tip section to a stiffer main body, with the resultant abrupt change in stiffness properties.

Referring back to FIGS. 2 and 3, the inner probe 110 comprises a reciprocating shaft 130 and an array 132 of tissue penetrating needle electrodes 134 extending from the distal end of the shaft 130. Like the rigid section 112 of the cannula 108, the inner probe shaft 130 is rigid and is composed of a suitable material, such as plastic or metal. To ensure that flexible section 116 of the cannula 108 maintains the required flexibility while the inner probe 110 is disposed within the cannula 108, the distal end of the inner probe shaft 130 should not extend distally beyond the distal end of the rigid cannula section 112. Alternatively, the inner probe shaft 130 can be composed of a semi-rigid material, such as, e.g., stainless steel braid, that when radially constrained by the inner surface of the cannula 108, provides the necessary columnar strength for the inner probe 110 to be distally pushed within the lumen 111 of the cannula 108. To facilitate coaxial movement between the inner probe shaft 130 and the cannula 108, the inner surface of the cannula 108 and/or the outer surface of the inner probe shaft 130 can be coated with a lubricious material. The electrode array 132 can be mounted anywhere on the inner probe shaft 130. However, the electrodes 134 will typically be fastened to the distal end of the shaft 130, though the proximal ends of the individual electrodes 134 can extend up to, or beyond, the proximal end of the shaft 130.

Each of the needle electrodes 134 is a small diameter metal element, which can penetrate into tissue as it is advanced into a target site within the target region. For example, each electrode 134 can be composed of a single wire that is formed from resilient conductive metals having a suitable shape memory. Many different metals such as stainless steel, nickel-titanium alloys, nickel-chromium alloys, and spring steel alloys can be used for this purpose. The wires may have circular or non-circular cross-sections, but preferably have rectilinear cross-sections. When constructed in this fashion, the needle electrodes 134 are generally stiffer in the transverse direction and more flexible in the radial direction. The circumferential alignment of the needle electrodes 134 within the cannula 108 can be enhanced by increasing transverse stiffness. Exemplary needle electrodes will have a width in the circumferential direction in the range of 0.2 mm to 0.6 mm, preferably from 0.35 mm to 0.40 mm, and a thickness, in the radial direction, in the range of 0.05 mm to 0.3 mm, preferably from 0.1 mm to 0.2 mm.

The distal ends of the needle electrodes 134 may be honed or sharpened to facilitate their ability to penetrate tissue. The distal ends of these needle electrodes 134 may be hardened using conventional heat treatment or other metallurgical processes. The needle electrodes 134 may be partially covered with insulation, although they will be at least partially free from insulation over their distal ends. The proximal ends of the needle electrodes 134 may be directly coupled to the proximal end of the inner probe shaft 130, or alternatively, may be indirectly coupled thereto via other intermediate conductors, such as RF wires (not shown). Optionally, the inner probe shaft 130 and any component between the shaft 130 and the needle electrodes 134 are composed of an electrically conductive material, such as stainless steel, and may therefore conveniently serve as intermediate electrical conductors.

As illustrated in FIG. 2, longitudinal translation of the inner probe shaft 130 in the proximal direction 136 relative to the cannula 108, retracts the electrode array 132 into the distal end of the cannula 108. When retracted within the cannula 108, the electrode array 132 is placed in a radially collapsed configuration, and each needle electrode 134 is constrained and held in a generally axially aligned position within the cannula 108 to facilitate its introduction into the tissue target site. The probe assembly 102 optionally includes a core member (not shown) mounted to the distal end of the inner probe shaft 130 and disposed within the center of the needle electrode array 132. In this manner, substantially equal circumferential spacing between adjacent needle electrodes 134 is maintained when the array is retracted within the central lumen 111.

As illustrated in FIG. 3, longitudinal translation of the inner probe shaft 130 in the distal direction 138 relative to the cannula 108 deploys the electrode array 132 out of the distal end of the cannula 108. As will be described in further detail, manipulation of the handle assembly 106 will cause the inner probe shaft 130 to longitudinally translate to alternately retract and deploy the electrode array 132. When deployed from the cannula 108, the electrode array 132 is placed in a three-dimensional configuration that usually defines a generally spherical or ellipsoidal volume having a periphery with a maximum radius in the range of 0.5 cm to 4 cm. The needle electrodes 134 are resilient and pre-shaped to assume a desired configuration when advanced into tissue. In the illustrated embodiment, the needle electrodes 134 diverge radially outwardly from the cannula 108 in a uniform pattern, i.e., with the spacing between adjacent needle electrodes 134 diverging in a substantially uniform pattern or symmetric pattern or both. In the illustrated embodiment, the needle electrodes 134 evert proximally, so that they face partially or fully in the proximal direction 136 when fully deployed. In exemplary embodiments, pairs of adjacent needle electrodes 134 can be spaced from each other in similar or identical, repeated patterns that can be symmetrically positioned about an axis of the inner probe shaft 130.

It will be appreciated by one of ordinary skill in the art that a wide variety of patterns can be used to uniformly cover the region to be treated. It should be noted that a total of eight needle electrodes 134 are illustrated in FIGS. 1-3. Additional needle electrodes 134 can be added in the spaces between the illustrated electrodes 134, with the maximum number of needle electrodes 134 determined by the electrode width and total circumferential distance available. Thus, the needle electrodes 134 could be quite tightly packed.

Referring back to FIG. 1, the steerable handle assembly 106 is mounted to the proximal ends of the cannula 108 and inner probe 110 (shown in phantom) and serves to conveniently allow the physician to alternately deploy and retract the electrode array 132. Specifically, the handle assembly 106 comprises a distal handle member 140 mounted to the proximal end of the rigid cannula section 112 and a proximal handle member 142 slidably engaged with the distal handle member 140 and mounted to the proximal end of the inner probe shaft 130. The proximal handle member 140 also comprises an electrical connector 144, which electrically couples the RF generator 104 to the proximal ends of the needle electrodes 134 (or alternatively, the intermediate conductors) extending through the inner probe shaft 130. The handle assembly 106 can be composed of any suitable rigid material, such as e.g., metal, plastic, or the like.

The handle assembly 106 also serves to conveniently allow the physician to selectively deflect the tissue penetrating tip 114 of the cannula 108, as shown in FIGS. 4 and 5 (or in the alternative embodiments, FIGS. 7, 8, 10, and 11). Specifically, the previously described steering assembly 122 is incorporated into the distal handle member 140 of the handle assembly 106. The steering assembly 122 includes a rotating cam wheel 146 (shown in phantom) and an external steering level or control 148 that rotates the cam wheel 146. The steering assembly 122 further comprises left and right steering wires 150 and 152, which extend along the associated left and right side surfaces of the cam wheel 146 and through steering lumens 154 and 156 contained within the cannula 108 (FIGS. 2, 3, 6, and 9).

The manner of mounting the steering wires 150 and 152 at the distal end of the cannula 108 will depend upon the specific structure of the intermediate flexible section 116. For example, in the case of the relatively short polymeric flexible section 116(1) illustrated in FIGS. 2 and 3, the distal ends of the steering wires 150 and 152 can be threaded through the steering lumens 154 and 156 within the flexible section 116 and connected to the tissue penetrating tip 114 using suitable means, such as welding. In the case of the segmented flexible section 116(2) illustrated in FIG. 6, the distal ends of the steering wires 150 and 152 can be threaded through opposing bores 158 and 160 (shown in FIG. 6A) within each rigid segment 124 (which when combined, forms the distal portion of the steering lumens 150 and 152) and connected to either the last rigid segment 124 or the tissue penetrating tip 114 using suitable means, such as welding. In this case of the reinforced polymeric flexible section 116(3) illustrated in FIG. 9, the distal ends of the steering wires 150 and 152 can be threaded through the steering lumens 154 and 156 within the tubular structure 126 and suitably connected to the outsides of the leaf springs 128 and 130.

Whichever flexible section 116 is used, manipulation of the steering level 148 causes the tissue penetrating tip 114 of the cannula 108 to deflect left or right, as shown in FIGS. 2 and 3 (or alternatively, FIGS. 7, 8, 10, and 11), thereby providing the cannula 108 with bi-directional steering capability. By rotating the distal handle member 140, thereby rotating the tissue penetrating tip 114 of the cannula 108, and by manipulating the steering lever 148, it is possible to maneuver the tissue penetrating tip 114 in virtually any direction. Alternatively, more steering wires and associated cams can be added to provide additional directionality to the tissue penetrating tip 114. Even more alternatively, only a single steering wire may be used to provide the cannula 108 with unidirectional steering capability. Additional details on the type of steering mechanism illustrated in FIG. 1 can be found in U.S. Pat. No. 5,363,861, which is hereby fully and expressly incorporated herein by reference. Other types of steering mechanisms are described in U.S. Pat. Nos. 6,033,378, 5,891,088, 5,531,686, 5,456,664, and 5,395,327, which are hereby fully and expressly incorporated herein by reference.

Alternatively, rather than using a steering wire-based steering assembly, a shape-memory steering system may be utilized. For example, instead of steering wires, shape-memory linkages (not shown) can be disposed through the steering lumens 154 and 156 of the cannula 108. The proximal ends of the shape memory linkages can be electrically stimulated to selectively active the linkages, thereby causing the flexible section 116 of the cannula 108 to flex one way or the other.

It should be noted that although the use of a steering assembly 122 is preferred in order to provide the probe assembly 102 with active steering capability, in some cases, the use of a steering assembly 122, along with the corresponding steering wires 150 and 152, may be foregone. For example, the beveled edge of the tissue penetrating tip 114 will tend to laterally bias the cannula 108 as it is advanced through tissue. The flexible section 116 will tend to magnify this bias, so that the tissue penetrating tip 114 will naturally angulate relative to the rigid cannula section 112 as the cannula 108 is advanced through tissue.

In the illustrated embodiment, the RF current is delivered to the electrode array 132 in a mono-polar fashion. Therefore, the current will pass through the electrode array 132 and into the target tissue, thus inducing necrosis in the tissue. To this end the electrode array 132 is configured to concentrate the energy flux in order to have an injurious effect on tissue. However, there is a dispersive electrode (not shown) which is located remotely from the electrode array 132, and has a sufficiently large area—typically 130 cm² for an adult—so that the current density is low and non-injurious to surrounding tissue. In the illustrated embodiment, the dispersive electrode may be attached externally to the patient, using a contact pad placed on the patient's skin. In a mono-polar arrangement, the needle electrodes 134 are bundled together with their proximal ends having only a single layer of insulation over the entire bundle.

Alternatively, the RF current is delivered to the electrode array 132 in a bipolar fashion, which means that current will pass between “positive” and “negative” electrodes 134 within the array 132. In a bipolar arrangement, the positive and negative needle electrodes 134 will be insulated from each other in any regions where they would or could be in contact with each other during the power delivery phase.

The probe assembly 102 may optionally have active cooling functionality, in which case, a heat sink (not shown) can be mounted within the distal end of the cannula 108 in thermal communication with the electrode array 132, and cooling and return lumens (not shown) can extend through the cannula 108 in fluid communication with the heat sink to draw thermal energy away back to the proximal end of the cannula 108. In this case, a pump assembly (not shown) can be provided to convey a cooling medium through the cooling lumen to the heat sink, and to pump the heated cooling medium away from the heat sink and back through the return lumen. Further details regarding active cooling of the electrode array 132 are disclosed in co-pending U.S. application Ser. No. ______ (Bingham & McCutchen Docket No. 24728-7011), which is hereby fully and expressly incorporated herein by reference.

As previously noted, the RF generator 104 is electrically connected, via the generator connector 104, to the handle assembly 106, which is directly or indirectly electrically coupled to the electrode array 132. The RF generator 104 is a conventional RF power supply that operates at a frequency in the range of 200 KHz to 1.25 MHz, with a conventional sinusoidal or non-sinusoidal wave form. Such power supplies are available from many commercial suppliers, such as Valleylab, Aspen, and Bovie. Most general purpose electro-surgical power supplies, however, operate at higher voltages and powers than would normally be necessary or suitable for controlled tissue ablation.

Thus, such power supplies would usually be operated at the lower ends of their voltage and power capabilities. More suitable power supplies will be capable of supplying an ablation current at a relatively low voltage, typically below 150V (peak-to-peak), usually being from 50V to 100V. The power will usually be from 20 W to 200 W, usually having a sine wave form, although other wave forms would also be acceptable. Power supplies capable of operating within these ranges are available from commercial vendors, such as RadioTherapeutics of San Jose, Calif., which markets these power supplies under the trademarks RF2000™ (100 W) and RF3000™ (200 W).

Although the tissue penetrating tip 114 has been previously described as integrally being formed with the distal end of the cannula 108, the tissue penetrating tip can be located on a separate structure, such as a trocar. For example, referring to FIG. 12, another probe assembly 202 that can be used in the tissue ablation system 100 is shown. The probe assembly 202 comprises a cannula 208, and a separate inner probe 210 and trocar 211, which can be selectively and alternately inserted in and removed from the cannula 208. Like the previously described cannula 108, the cannula 208 comprises a rigid section 212. The cannula 208 also comprises a segmented flexible section 216 similar to the segmented flexible section 116(2) illustrated in FIGS. 6-8. Alternatively, a flexible section 216 similar to the polymeric flexible section 116(3) illustrated in FIGS. 9-11 can be used. It can be appreciated that whichever flexible section is used, it will now form the distal end of the cannula 208, rather than forming an intermediate section thereof.

The inner probe 210 is similar to the previously described inner probe 110 in that it comprises a shaft 230 and a distally mounted needle electrode array 232. The inner probe 210 differs, however, in that can be selectively inserted into and removed from the cannula 208. The trocar 211 comprises a shaft 213 and a rigid tissue penetrating tip 214 mounted to the distal end of the shaft 213. The tissue penetrating tip 214 is constructed similarly to the previously described tissue penetrating tip 114. Additionally, the tissue penetrating tip 214 can be faceted for ultrasound visualization. When the trocar 211 is fully inserted into the cannula 208, the tissue penetrating tip 214 will distally protrude from the distal end of the flexible section 216. Thus, the cannula 208, with the aid of the trocar 211, will be able to penetrate and advance through tissue. The trocar shaft 213 is laterally flexible, yet exhibits relatively high columnar strength when constrained within the cannula 208. As such, the distal end of the of the trocar 211 will not significantly inhibit bending of the flexible section 216, and thus deflection of the tissue penetrating tip 214. For example, the trocar shaft 213 can be composed of a semi-rigid material, such as, e.g., stainless steel braid, that when radially constrained by the inner surface of the cannula 208, provides the necessary columnar strength for the trocar 211 to be distally pushed within the cannula 208. The inner probe shaft 230 can be similarly constructed to provide laterally flexibility and columnar strength thereto.

The probe assembly 202 lastly includes a steerable handle assembly 206 that is similar to the previously described steerable handle assembly 106, with the exception that it does not form an integrated assembly until either the trocar 211 or the inner probe 210 is fully inserted into the cannula 208. Specifically, the handle assembly 206 comprises a distal handle member 240 mounted to the proximal end of the cannula 208, and separate proximal handle members 242 and 243 that are respectively mounted to the proximal ends of the inner probe shaft 230 and the trocar shaft 213. The proximal handle members 242 and 243 are configured, such that once the respective inner probe 210 or trocar 211 is fully inserted into the cannula 208, they are slidable disposed within the distal handle member 240. Either or both of the proximal handle members 242 and 243 can include a locking mechanism, such as a luer lock (not shown), so that the inner probe 210 and trocar 211 can releasably engage the cannula 208. The handle member 242 includes the previously described RF connector 144 for electrical coupling to the RF generator 104 (shown in FIG. 1). The handle assembly 206 comprises the previously described steering assembly 122, which is incorporated into the distal handle member 240. Thus, the distal ends of the steering wires 150 and 152 (not shown in this embodiment) will be mounted to the last rigid segment of the flexible section 216. As such, manipulation of the steering assembly 122 will bend the distal end of the cannula 208, and specifically the flexible section 216, and thus deflect either the tissue penetrating tip 214 or the needle electrode array 232, depending on which of the trocar 211 and inner probe 210 is inserted within the cannula 208.

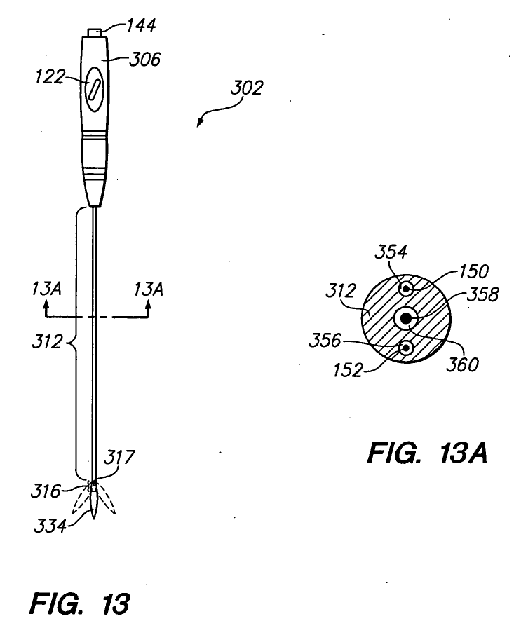

Although the probe assemblies 102 and 202 have been previously described as employing multiple needle electrodes, other types of electrode arrangements can be envisioned. For example, referring to FIG. 13, a probe assembly 302 employing a single electrode is illustrated. The probe assembly 202 comprises a rigid section 312, a rigid tissue penetrating needle electrode 334, and an intermediate flexible section 316 mounted between the rigid section 312 and the needle electrode 334. The rigid section 312 is composed of a suitable material, such as, e.g., plastic or metal. The needle electrode 334 is composed of a suitably conductive, yet biocompatible, material, such as stainless steel or copper. In the embodiment illustrated in FIG. 13, the flexible section 316, like the flexible section 116(1) illustrated in FIGS. 2 and 3, comprises a relatively short tubular structure 317 that is suitably bonded between the distal end of the rigid section 312 and the proximal end of the needle electrode 234. Alternatively, a segmented flexible structure, such as that illustrated in FIGS. 6-8, or a reinforced polymeric flexible structure, such as that illustrated in FIGS. 9-11, can be used.

The probe assembly 302 further comprises a handle assembly 306 that includes the previously described steering assembly 122 and RF connector 144. Steering wires 150 and 152 extend from the steering assembly 122 through steering wire lumens 354 and 356 extending through the rigid section 312, distally terminating at the proximal end of the needle electrode 134 using suitable means, such as welding. An RF wire 358 extends from the RF connector 144, and through an RF wire lumen 360 extending through the rigid section 312. Thus, manipulation of the steering assembly 122 causes the flexible section 316 to bend, and thus the needle electrode 134 to deflect. Like the steering wires 150 and 152, the RF wire 360 also terminates at the proximal end of the needle electrode 334 using suitable means, such as welding. Optionally, the needle electrode 334 may have active cooling functionality, in which case, cooling and return lumens (not shown) can be provided through the rigid section 312 and needle electrode 334. The handle assembly 306 can be modified to include input and output ports (not shown) that can be connected to a pump assembly (also not shown) for circulating a cooled medium through the needle electrode 334.

Having described the structure of the tissue ablation system 100, its operation in treating targeted tissue will now be described. The treatment may be located anywhere in the body where hyperthermic exposure may be beneficial. Most commonly, the treatment region will comprise a solid tumor within organ of the body, such as the liver, kidney, pancreas, breast, and prostate (not accessed via the urethra). The volume to be treated will depend on the size of the tumor or other lesion, typically having a total volume from 1 cm³ to 150 cm³, and often from 2 cm³ to 35 cm³. The treatment region can also include regions that require soft-tissue ablation such as in procedures involved with orthopedics, pain-management (e.g., spinal disk shrinkage), trans-vaginal ablation of uterine fibroids, fallopian tube closure for sterilization, etc. The peripheral dimensions of the treatment region will sometimes be regular, such as, for example, when they are spherical or ellipsoidal. However, the dimensions will more usually be irregular. The target region may be identified prior to treatment using conventional imaging techniques that are capable of elucidating a target tissue, such as a tumor. These imaging techniques include ultrasonic scanning, MRI, CT scanning, fluoroscopy, and nuclear scanning using radio-labeled tumor specific probes.

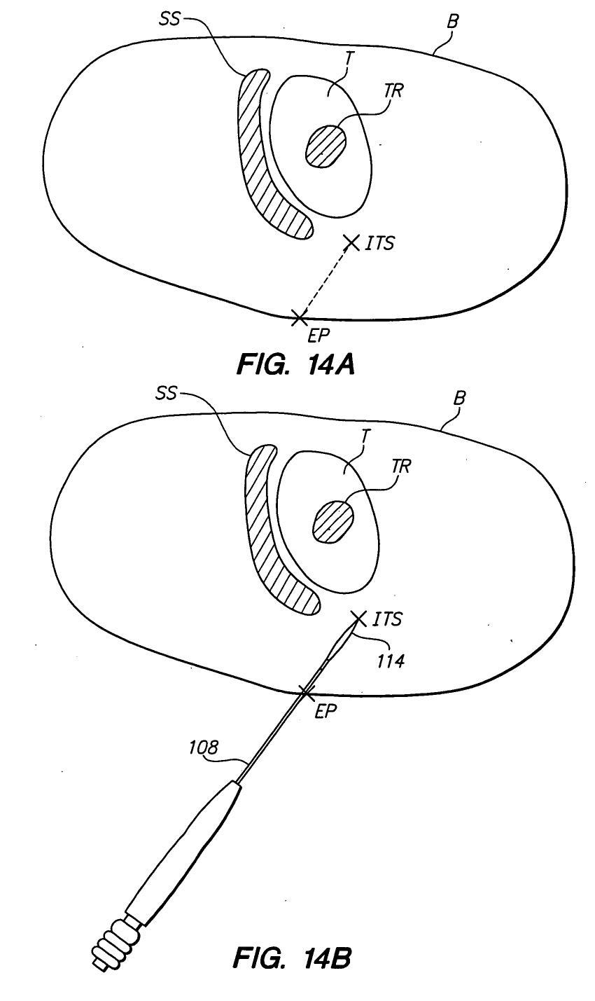

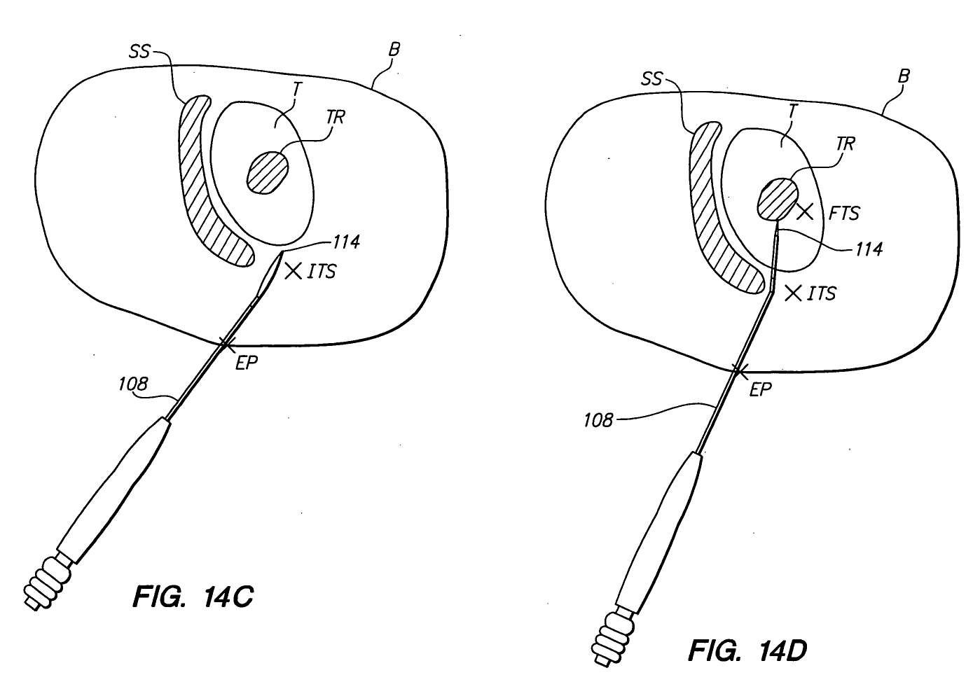

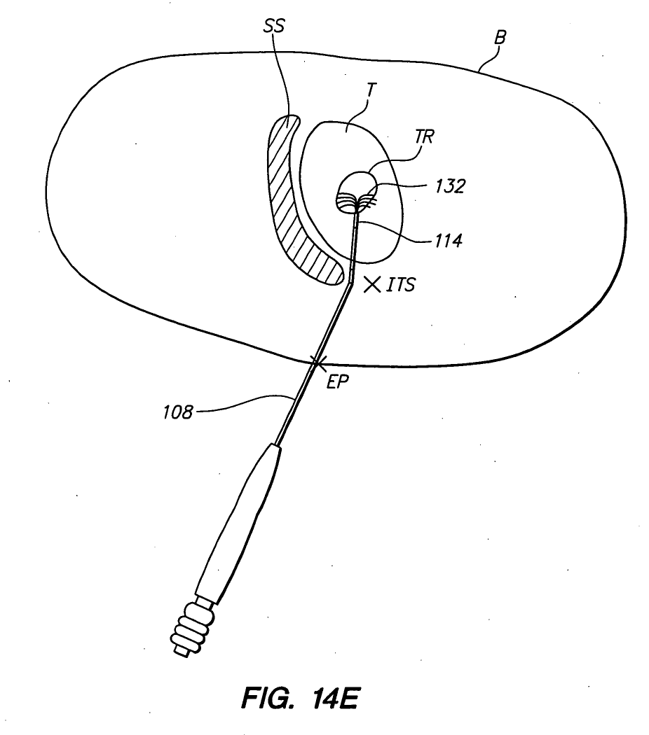

Referring now to FIGS. 14A-14E, the operation of the tissue ablation system 100 is described in treating a treatment region TR, such as a tumor, within a tissue T, e.g., an organ, located in a patient's body B. The tissue T prior to treatment is shown in FIG. 14A. As illustrated, a sensitive structure SS is located adjacent the treatment region TR. After identifying the treatment region TR and the sensitive structure SS using a suitable imaging means, the physician plans an entry track that would avoid the sensitive structure SS. Normally, using a straight-line approach, this entry track may not be optimum. Using the steerable probe assembly 102, however, the physician can plan an optimum entry track notwithstanding that the sensitive structure SS may lie directly between the treatment region TR and an entry point EP. For example, in the illustrated embodiment, the physician plans an entry track ET that bypasses the sensitive structure SS, extending from the entry point EP to a initial target site ITS just distal to the sensitive structure SS.

After the entry track ET has been planned, the cannula 108 is introduced within the tissue T, so that the tissue penetrating tip 114 is located at the initial target site ITS, as shown in FIG. 14B. This can be accomplished using any one of a variety of techniques. In this case, because the cannula 108 has sufficient columnar strength and carries the tissue penetrating tip 114, it and the inner probe 110 may be introduced to the initial target site ITS percutaneously directly through the patient's skin or through an open surgical incision. Alternatively, the probe assembly 202 with the single needle electrode 334 can similarly be introduced into to the initial target site ITS. More alternatively, if the probe assembly 202 with the trocar 211 is used, the cannula 208 may be introduced to the initial target site ITS with the trocar 211 fully inserted within the cannula 208 and locked in place, so that the tissue penetrating tip 214 of the trocar 211 extends from the distal end of the cannula 208 as it is advanced through the tissue T. Once properly placed, the trocar 211 can then be exchanged for the inner probe 210. More alternatively, a conventional sheath and the trocar 211 can be used to initially access the initial target site ITS under ultrasonic or conventional imaging, with the trocar 211 then removed to leave an access lumen through the sheath. The cannula 208, with the inner probe 210, can then be introduced through the sheath lumen, so that the distal end of the cannula 208 advances from the sheath into the initial target site ITS.

After the cannula 108 is properly placed at the initial target site ITS, the steering assembly 122 is manipulated, so that the tissue penetrating tip 114 deflects towards the treatment region TR, as illustrated in FIG. 14C. Proper deflection can be confirmed with the use of conventional imaging techniques and/or placement of a marker on the handle assembly 106 that indicates a reference rotational orientation of the cannula 108. Once the proper deflection of the tissue penetrating tip 114 is achieved, the cannula 108 is then further advanced, so that the tissue penetrating tip 114 advances towards the treatment region TR to a final target site FTS, as illustrated in FIG. 14D. If the initial deflection angle is incorrect, the deflection of the tissue penetrating tip 114 can be corrected, such that the tissue penetrating tip 114 can be resteered towards the final target site FTS. Thus, the active steering capability of the probe assembly 102 conveniently allows the physician flexibility in choosing the path to the final target site FTS. It should be noted that, in the case where the initial target site ITS is coincident with, or very near, the final target site FTS, the cannula 108 need not to be advanced much further, or not any further, than the initial target site ITS.

Alternatively, if there is no steering assembly 122 to actively steer the penetrating tip 114, the cannula 108 can simply be advanced, so that the compressive axial force caused by the tissue resistance flexes the tissue penetrating tip 114 towards the treatment region TR. Proper trajectory of the tissue penetrating tip 114 is preferably accomplished the first time that the cannula 108 is advanced, since no means for easily correcting the trajectory will be available in this case.

After the cannula 108 has reached the final target site FTS, the inner probe shaft 130 is distally advanced to deploy the electrode array 132 radially outward from the distal end of the cannula 108, as shown in FIG. 14E. The inner probe shaft 130 will be advanced sufficiently, so that the electrode array 132 fully everts in order to circumscribe substantially the entire treatment region TR.

The RF generator 104 is then connected to the RF connector 144 on the handle assembly 106, and operated to create a lesion within the treatment region TR. If the treatment region TR is significantly larger than the maximum area that the electrode array 132 is capable of circumscribing, the cannula 108, with the electrode array 132 retracted, will need to be repositioned, and the electrode array 132 redeployed in a different position within the treatment region TR. The RF generator 104 will then be operated again to create a second lesion in the treatment region TR. These steps will be repeated as necessary in order to ablate the entirety of the treatment region TR.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A probe assembly for ablating tissue, comprising:
an elongated shaft having a rigid section and a flexible section distal to the rigid section;
a rigid tissue penetrating element distally associated with the flexible section of the shaft; and
one or more ablative elements distally associated with the flexible section of the shaft.

2. The probe assembly of claim 1, further comprising a trocar having a distal end, wherein the tissue penetrating element is disposed on the distal end of the trocar, the shaft comprises a cannula having a lumen, and the trocar can be reciprocatably disposed within the cannula lumen.

3. The probe assembly of claim 1, wherein the tissue penetrating element is distally mounted to the flexible section of the shaft.

4. The probe assembly of claim 1, wherein the shaft is a cannula having a lumen, and the one or more ablative elements are distally deployable from the cannula lumen.

5. The probe assembly of claim 1, wherein the one or more ablative elements are disposed on the tissue penetrating element.

6. The probe assembly of claim 1, wherein the flexible section comprises a relatively short polymeric tubular structure.

7. The probe assembly of claim 1, wherein the flexible section comprises a polymeric tubular structure with one or more stiffening members disposed along an axis of the tubular structure.

8. The probe assembly of claim 1, wherein the flexible section of the shaft comprises a plurality of rigid segments.

9. The probe assembly of claim 1, wherein the tissue penetrating element is faceted for ultrasound visualization.

10. The probe assembly of claim 1, further comprising a steering mechanism for actively flexing the flexible section of the shaft.

11. The probe assembly of claim 10, wherein the steering mechanism is configured to flex the flexible section of the shaft in a single direction.

12. The probe assembly of claim 10, wherein the steering mechanism is configured to flex the flexible section of the shaft in multiple directions.

13. The probe assembly of claim 10, wherein the steering mechanism comprises one or more wires mounted to the flexible section of the shaft or the tissue penetrating element.

14. The probe assembly of claim 13, wherein the flexible section comprises a resilient stiffening member, and the one or more wires are mounted to the stiffening member.

15. The probe assembly of claim 10, wherein the steering mechanism comprises a plurality of wires mounted to the flexible section of the shaft or the tissue penetrating element.

16. The probe assembly of claim 10, wherein the steering mechanism comprises a one or more shape-memory linkages.

17. The probe assembly of claim 1, wherein the one or more ablative elements comprises one or more ablation electrodes.

18. The probe assembly of claim 1, wherein the tissue penetrating element and the one or more ablative elements are formed by the same structure.

19. A method of treating a tissue region within a patient, comprising:
inserting a medical probe into the body of the patient via an entry point;
identifying a sensitive anatomical structure located between the entry point and the tissue region;
advancing the medical probe, such that a distal end of the medical probe bypasses the sensitive anatomical structure;
bending the distal end of the medical probe while the medical probe is within the body of the patient;
placing one or more ablative elements associated with the distal end of the medical probe adjacent the tissue region; and
ablating the tissue region with the one or more ablative elements.

20. The method of claim 19, wherein the distal end of the medical probe is bent after the distal end of the medical probe bypasses the sensitive anatomical structure.

21. The method of claim 19, wherein the medical probe is percutaneously inserted into the body of the patient via the entry point.

22. The method of claim 19, wherein the medical probe is laparoscopically inserted into the body of the patient via the entry point.

23. The method of claim 19, further comprising advancing the medical probe again after the distal end of the medical probe has been bent, such that the distal end of the medical probe is adjacent the tissue region.

24. The method of claim 19, wherein the medical probe is initially advanced to an initial target site, and then advanced again to a final target site after the distal end of the medical probe has been bent.

25. The method of claim 19, wherein the tissue region is a tumor.

26. The method of claim 19, wherein the sensitive anatomical structure is an organ.

27. The method of claim 19, wherein the sensitive anatomical structure is a blood vessel.

28. The method of claim 19, wherein the one or more ablative elements are deployed from the medical probe.

29. The method of claim 19, wherein the one or more ablative elements are affixed to the distal end of the medical probe.

30. The method of claim 19, wherein the distal end of the medical probe is bent using a steering mechanism.

31. The method of claim 19, wherein the tissue region is ablated using radio frequency (RF) energy.

32. The method of claim 19, wherein the one or more ablative elements are embedded within the tissue region.
